(19) United States
(12) Patent Application Publication
(10) Pub. No.: US 2023/0265868 A1
(43) Pub. Date: Aug. 24, 2023

(54) HYDRAULIC LOCKING DEVICE FOR AN EXOSKELETON JOINT AND EXOSKELETON JOINT

(71) Applicant: HAWE Hydraulik SE, Aschheim (DE)

(72) Inventors: Christian Hart, Aschheim (DE); Julian Weber, Aschheim (DE); Steffen Marx, Aschheim (DE)

(21) Appl. No.: 18/167,172

(22) Filed: Feb. 10, 2023

(30) Foreign Application Priority Data
Feb. 21, 2022 (DE) 10 2022 201 764.1

(51) Int. Cl. F15B 15/26; F15B 15/14

(52) U.S. Cl. CPC F15B 15/26; F15B 15/1423; F15B 2015/268; F15B 2015/267

(57) ABSTRACT

A hydraulic locking device for an exoskeleton joint with an extendable and retractable hydraulic cylinder, a tank and a switching valve. The hydraulic cylinder is connected to the tank via a line arrangement and the switching valve is disposed in the line arrangement. The switching valve is switchable between a release position and a blocking position. The hydraulic cylinder is freely movable in the release position of the switching valve, and the switching valve prevents retraction of the hydraulic cylinder in the blocking position. The hydraulic locking device further comprises a housing, and the hydraulic cylinder is a plunger cylinder with a plunger cylinder housing and a plunger piston movably arranged in the plunger cylinder housing, the plunger cylinder being arranged within the housing. Further, an exoskeleton joint having such a hydraulic locking device is disclosed.

### CROSS REFERENCE TO RELATED APPLICATION

This application claims priority to German Application 10 2022 201 764.1, filed Feb. 21, 2022, which is hereby incorporated by reference in its entirety.

### FIELD OF THE INVENTION

The present invention relates to a hydraulic locking device for an exoskeleton joint, and to an exoskeleton joint having such a hydraulic locking device.

### BACKGROUND OF THE INVENTION

Exoskeletal joints are used to join sections of an exoskeleton together in an articulated manner. Such exoskeletons serve as an external support structure and are regularly used as load support devices for persons, for example to reduce the person's energy requirements, to reduce physical stress or as part of medical rehabilitation therapies. It is also conceivable that the device could be used as a training device during stays in space in order to specifically train the muscle groups that are not used due to the weightlessness.

Preferably, exoskeletons are used for the lower human extremities, which support a weight relative to the ground during a stance phase and are free to move during a swing phase. In other words, the known exoskeletons provide an external support structure for the legs of a human user, in that a weight to be carried by the user (for example, a backpack) is supported by the exoskeleton relative to the ground during the stance phase of the user. During the swing phase—i.e. the movement of a leg—no support is provided. Instead, free movement is provided here.

For this purpose, it is necessary that the exoskeleton joints used can be moved with as little friction as possible by the user's muscle power during the swing phase and are locked during the stance phase. Such an exoskeleton joint can be an exoskeleton knee joint, for example.

Hydraulic locking devices are regularly used to block the exoskeleton joint. These hydraulic locking devices usually have an extendable and retractable hydraulic cylinder, a tank and a switching valve. The hydraulic cylinder is provided to follow the movement the exoskeleton joint by extending and retracting and to block the exoskeleton joint in that the hydraulic cylinder is hydraulically blocked.

For this purpose, the hydraulic cylinder is connected to the tank via a line arrangement and the switching valve is disposed in the line arrangement. The switching valve can be switched between a release position and a blocking position. In the release position of the switching valve, the hydraulic cylinder is freely movable. Thus, during the swing phase, the switching valve is in the release position so that the hydraulic cylinder can follow the rotation of the exoskeleton joint by moving in or out. In the stance phase, the switching valve is switched to the blocking position so that a connection between the hydraulic cylinder and the tank is interrupted. Acceleration sensors, contact sensors or even pressure sensors can be provided, for example, to detect the stance phase or the swing phase.

FIG. 1 shows a hydraulic circuit diagram of a known prior art hydraulic locking device 100, which is used in an exoskeleton joint. The hydraulic locking device 100 comprises a hydraulic cylinder configured as a differential cylinder 102, which is connected to a tank configured as an accumulator 106 via a line arrangement 104. The differential cylinder 102 comprises a cylinder housing 108 and a piston 110 movably disposed in the cylinder housing 108. The piston 110 separates a piston chamber 112 from a rod chamber 114. The line arrangement 104 includes a first branch 116 connected to the piston chamber 112. A second branch 118 of the line arrangement 104 is connected to the rod chamber 114. A switching valve 120 is disposed in the first branch 116, which is switchable between a release position FS and a blocking position SS. When current is applied to the switching valve 120, the switching valve 120 switches from the release position FS to the blocking position SS against a spring force. Furthermore, the hydraulic locking device 100 comprises a short-circuit line 122 bypassing the switching valve 120 between the first branch 116 and the second branch 118. A spring-loaded check valve 124 is disposed in the short-circuit line 122, which opens in the direction of flow from the rod chamber 114 to the piston chamber 112.

The switching valve 120 is actuated by a control (not shown) in such a way that it is in the blocking position SS in the stance phase and thus blocks the first branch 116. In this case, the piston 110 is blocked against retraction, since no hydraulic fluid can be displaced from the piston chamber 112 to the tank 106. Extension of the piston 110 is still possible, as hydraulic fluid can be sucked from the tank 106 via the check valve 124. In the case of an exoskeleton knee joint, this practically means that the leg supported by the exoskeleton can be further extended, but cannot be bent above the knee. The opening angle of the exoskeleton knee joint can therefore be increased, but not decreased. This can ensure that full extension of the leg is achieved even if the switching valve 120 is already in the blocking position SS.

An exoskeleton joint with such a hydraulic locking device is known, for example, from EP 2 687 339 B1. In the prior art, the differential cylinder is regularly connected in an articulated manner to the upper leg of the exoskeleton and the lower leg of the exoskeleton and is blocked in the stance phase via the switching valve.

A disadvantage of the known solution is that the hydraulic cylinder interferes with the user when sitting down, as it is mounted directly behind the knee joint. Solutions are therefore known from the prior art in which the hydraulic cylinders are arranged on the side of the knee joint. However, the problem here is that the exoskeleton is then very large at the side and the hydraulic cylinders can be damaged more easily, for example by getting caught on rigid structures. Furthermore, laterally arranged hydraulic cylinders also pose the problem that a rotational moment is introduced, which must be compensated. Laterally arranged hydraulic cylinders can also be a nuisance when walking, as they are then located in the swing area of the arms. Furthermore, the hydraulic locking devices in the known solutions take up a relatively large amount of space and are also heavy.

### SUMMARY OF THE INVENTION

It is therefore the objective of the present invention to provide a simply constructed hydraulic locking device for an exoskeleton joint, which takes up little installation space, is protected against damage, does not impair the user as far as possible and is weight-reduced.

The problem is solved with a hydraulic locking device for an exoskeleton joint according to the embodiments of the present invention disclosed herein.

The hydraulic locking device for an exoskeleton according to the invention is characterized over hydraulic locking devices known in the prior art in particular by the fact that the hydraulic locking device has a housing and the hydraulic cylinder is a plunger cylinder with a plunger cylinder housing and a plunger piston movably arranged in the plunger cylinder housing. According to the invention, the plunger cylinder is disposed inside the housing.

Plunger cylinders are single-acting cylinders and are also known as plunger piston cylinders. Plunger cylinders do not have an actual piston, but the piston rod serves as the piston. Due to the external resetting force, caused by the muscle power of the user, during the swing phase, the disadvantage of the more complex resetting with plunger cylinders is also irrelevant. Overall, the use of a plunger cylinder results in a more favorable mechanical efficiency than the conventionally used differential cylinders. As a result, the plunger cylinder can be of relatively small design and therefore also of reduced weight. In addition, disposition within the housing also provides protection against damage, and a corresponding arrangement directly in the exoskeleton joint can prevent the user from being impaired by the hydraulic locking device.

Preferably, the hydraulic locking device has a rotary shaft and a lever. Preferably, the rotary shaft is rotatably mounted on the housing about a first axis of rotation and the plunger cylinder housing is rotatably mounted on the housing about a second axis of rotation. Preferably, the lever has a shaft coupling portion and a piston coupling portion, the shaft coupling portion being non-rotatably connected to the rotary shaft. Preferably, the piston coupling section is rotatably connected to the plunger.

The rotary shaft transmits the rotary motion to the plunger via the lever. When the switching valve is in the blocking position, the plunger is secured against retraction. Due to the direct connection via the lever, further rotation of the rotary shaft is thus prevented. A possible force is thus supported via the rotary shaft, the lever and the plunger cylinder. It is preferably if the first axis of rotation is parallel to the second axis of rotation.

Preferably, the rotary shaft is rotatable between a first end position and a second end position, a dead point position being located between the first end position and the second end position. The plunger is preferably extended in the first end position and moves into the dead point position upon rotation of the rotary shaft. Upon rotation from the dead point position to the second end position, the plunger preferably extends.

The first end position of the rotary shaft can correspond, for example, to the maximum possible angle of the exoskeleton joint. The second end position can thus correspond to the minimum possible angle of the exoskeleton joint, for example. Using the example of an exoskeleton knee joint, this means that the rotary shaft is in the first end position when the leg is largely or fully extended and in the second end position when the leg is largely or fully bent.

During a movement from the first end position towards the second end position, the plunger initially retracts. At the dead point position of the rotary shaft, which lies between the first end position and the second end position, the plunger is retracted to the maximum. During a further movement from the dead point position in the direction of the second end position, the plunger extends again. The direction of movement of the plunger is thus reversed. Thus, the entire maximum movement between the first end position and the second end position can be reproduced with a significantly smaller stroke of the plunger cylinder. This results in a particularly space-saving and weight-reduced design of the hydraulic locking device.

Preferably, the switching valve has a check valve that is active in the blocking position, with the check valve opening in the direction of flow from the tank to the plunger cylinder. The check valve thus allows the plunger cylinder to be refilled, so that the plunger piston can extend further. Retraction of the plunger piston is not possible when the switching valve is in the blocking position. Thus, the exoskeleton joint can also be opened further, although the switching valve is in the blocking position. Using the exoskeleton knee joint as an example, this means that the user's knee can be extended further even if the switching valve is already in the blocking position or the stance phase has been detected. However, it is not possible to bend the knee when the switching valve is in the blocking position, so that a possible load is still safely and reliably supported via the exoskeleton.

Alternatively the plunger cylinder has a check valve, the check valve opening in the direction of flow from the tank to the plunger cylinder. It is particularly preferable if the plunger piston has the check valve. This results in the same advantages as already described above, namely that extension of the plunger piston is also possible in the blocking position of the switching valve. In addition, the integration of the check valve into the plunger results in a particularly compact and lightweight design.

Preferably, the plunger piston and the plunger cylinder housing define a plunger chamber, the plunger chamber being connected to the tank via a pressure relief valve. The pressure relief valve acts as a safety valve by setting a maximum allowable pressure at the pressure relief valve. As soon as the pressure in the plunger chamber, and thus also at least in sections in the line arrangement, exceeds the maximum pressure set at the pressure relief valve, the pressure relief valve opens and the plunger chamber is relieved to the tank. In this way, damage to the hydraulic locking device can be effectively prevented.

The housing preferably forms the tank, with the plunger cylinder being disposed inside the tank. The plunger cylinder thus does not have to be elaborately sealed against leakage, because any leakage escapes directly into the tank. In addition, the stance phase is usually relatively short, so that a certain amount of leakage from the plunger cylinder over time can be tolerated without further ado. As a result, a high-pressure seal of the plunger piston relative to the plunger cylinder housing can be dispensed with, and only a gap seal needs to be provided.

Furthermore, this also eliminates the need to provide a large number of high-pressure seals throughout the hydraulic locking device. Instead, only one high-pressure seal needs to be provided between the plunger cylinder or plunger chamber and the switching valve, because this is the only section of the hydraulic locking device where pressures above atmospheric pressure can occur.

The solution of the problem is further achieved with an exoskeleton joint according to claim 9, which comprises a hydraulic locking device described above. The exoskeleton joint is preferably an exoskeleton knee joint. However, it is also conceivable that the exoskeleton joint is an exoskeleton hip joint, an exoskeleton elbow joint, an exoskeleton ankle joint, or an exoskeleton shoulder joint.

Preferably, the exoskeleton joint has a first receptacle disposed on the housing of the hydraulic locking device and a second receptacle movably mounted on the housing via a four-bar linkage. The four-bar linkage is preferably connected to the rotary shaft. For example, the upper leg of the exoskeleton may be attached to the first receptacle. The lower leg of the exoskeleton can be attached to the second receptacle, for example.

### THE BRIEF DESCRIPTION OF THE DRAWINGS

FIG. 1 is a hydraulic circuit diagram of a hydraulic locking device known from the prior art;

FIG. 2 is a hydraulic circuit diagram of a hydraulic locking device according to the invention in accordance with a first embodiment;

FIG. 3 is a hydraulic circuit diagram of a hydraulic locking device according to the invention in accordance with a second embodiment;

FIG. 4 is a hydraulic circuit diagram of a hydraulic locking device according to the invention in accordance with a third embodiment;

FIG. 5 is a first perspective view of a hydraulic locking device according to the invention;

FIG. 6 is the hydraulic locking device according to FIG. 5 in a second perspective view;

FIG. 7 is a partially exposed side view of the hydraulic locking device according to FIG. 5;

FIG. 8 is another partially exposed side view of the hydraulic locking device according to FIG. 5,

FIG. 9 is the view according to FIGS. 7 and 8, with the plunger cylinder shown in various positions,

FIG. 10 is a side view of an exoskeleton joint with a hydraulic locking device according to FIG. 2, and

FIG. 11 is a partial section through the exoskeleton joint shown in FIG. 10.

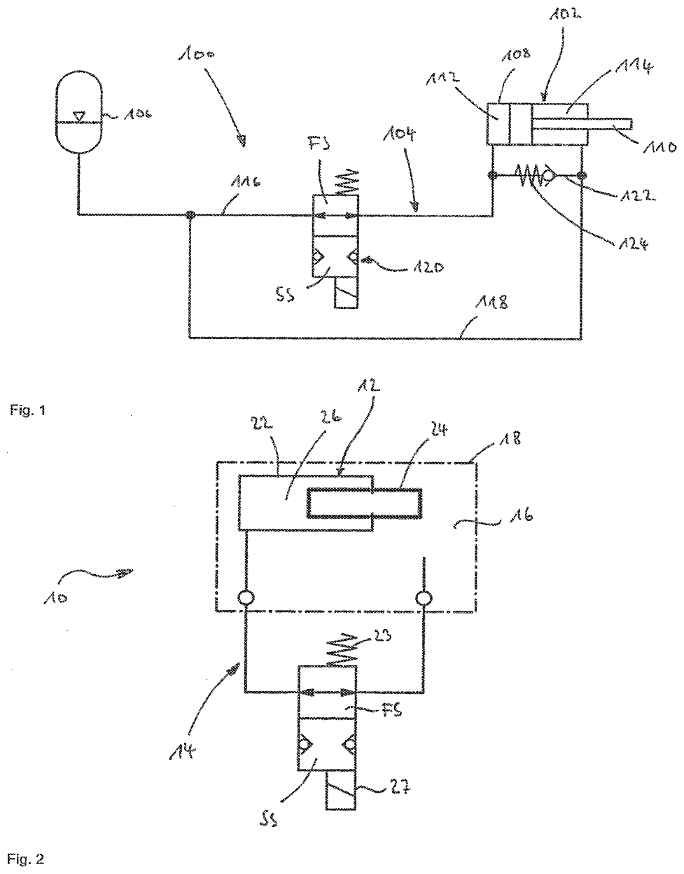

### DETAILED DESCRIPTION OF THE DRAWINGS

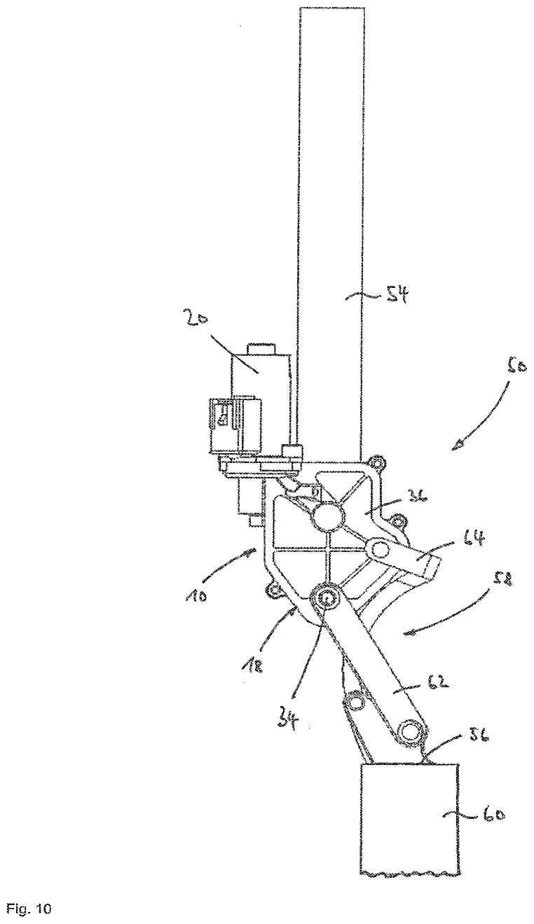

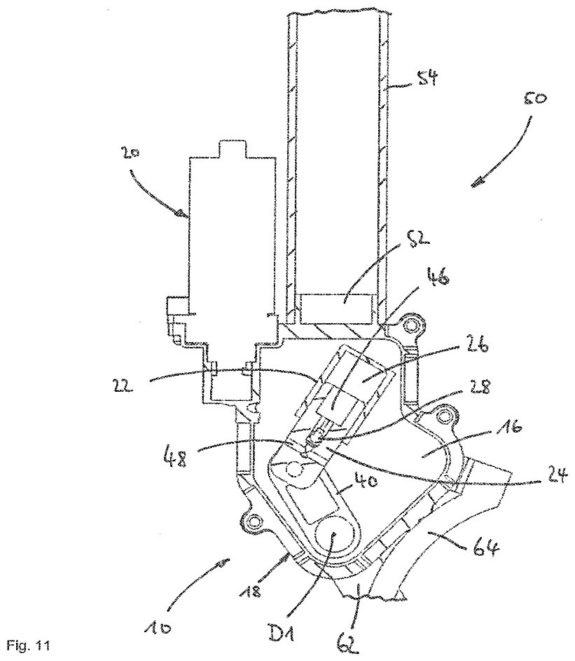

FIG. 2 shows a hydraulic circuit diagram of a hydraulic locking device 10 according to the invention for an exoskeleton joint 50 according to a first embodiment. The hydraulic locking device 10 can be used, for example, with an exoskeleton knee joint 50, as exemplified in FIGS. 10 and 11. The hydraulic locking device 10 comprises a hydraulic cylinder configured as a plunger cylinder 12, which is connected to a tank 16 via a line arrangement 14. Further, the hydraulic locking device 10 comprises a housing 18 forming the tank 16. The plunger cylinder 12 is disposed within the housing 18 and thus within the tank 16. Thus, the plunger cylinder 12 is under hydraulic fluid.

A switching valve 20 is disposed in the line arrangement 14, which can be switched between a release position FS and a blocking position SS. The switching valve 20 comprises a biasing element 23 which biases the switching valve into the release position FS. An actuating device 27 is actuated to switch the switching valve 20 to the blocking position SS. In this embodiment, the actuating device 27 is an electromagnet that is energized via a (not shown) higher-level control system.

The plunger cylinder 12 comprises a plunger cylinder housing 22 and a plunger piston 24 axially movable within the plunger cylinder housing 22. The plunger piston 24 and the plunger cylinder housing 22 define a variable volume plunger chamber 26.

If free movement is required, the switching valve 20 is in the release position FS and hydraulic fluid can be sucked from the tank 16 via the line arrangement 14 when the plunger piston 24 is extended. Accordingly, hydraulic fluid is displaced from plunger chamber 26 by an inward movement of plunger piston 24 and is directed into tank 16 via the line arrangement. As soon as the switching valve 20 is switched to the blocking position SS, the connection between the tank 16 and the plunger chamber 26 is blocked. The plunger piston 24 is then fixed in the position relative to the plunger cylinder housing 22, and cannot extend or retract.

As noted above, the plunger cylinder 12 is disposed entirely within the tank 16 so that any potential leakage from the plunger cylinder 12 is not problematic. Thus, the plunger cylinder 12 can be of simple construction without the need for a high pressure seal between the plunger piston 24 and the plunger cylinder housing 22. A gap seal is sufficient between the plunger piston 24 and the plunger cylinder housing 22. This is because possible leakage-induced movement of the plunger piston 24 despite the switching valve 20 being switched to the blocking position SS is tolerable when the hydraulic locking device 10 is used in an exoskeleton joint 50. The blocking of the movement of the plunger piston 24 serves to support a load, for example during a stance phase. However, this phase is very limited in time, so that some leakage, and thus some movement of the plunger piston 24, over this short period of time is insignificant.

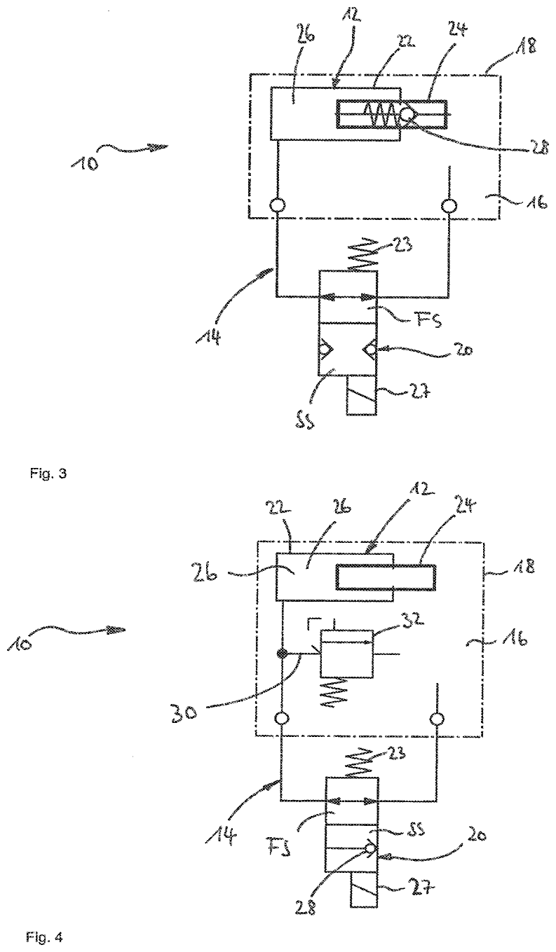

FIG. 3 shows a hydraulic circuit diagram of a second embodiment of a hydraulic locking device 10 according to the invention. The embodiment shown in FIG. 3 differs from the embodiment shown in FIG. 2 in that a check valve 28 is disposed in the plunger piston 24. The check valve 28 is a spring-loaded check valve 28 and it opens against the spring force in the direction of flow from the tank 16 to the plunger chamber 26.

In the blocking position SS of the switching valve 20, the plunger piston 24 is locked against retraction. However, the plunger piston 24 can continue to extend in the blocking position SS of the switching valve 20, since hydraulic fluid can be sucked into the plunger chamber 26 via the check valve 28. Using the example of the exoskeleton knee joint 50 described in more detail below, this means that the knee can be extended even further when the switching valve 20 is in the blocking position SS. However, bending of the knee is not possible because the plunger piston 24 is locked against retraction in the locking position SS of the switching valve 20.

The integration of the check valve 28 into the plunger piston 24 results in a particularly compact design of the plunger cylinder 12.

FIG. 4 shows a hydraulic circuit diagram of a third embodiment of a hydraulic locking device 10 according to the invention. The hydraulic locking device 10 according to the third embodiment differs from the hydraulic locking device 10 according to the second embodiment shown in FIG. 3, firstly, in that the check valve 28 is not disposed in the plunger piston 24, but in the switching valve 20. As shown, the check valve 28 is active in the blocking position SS of the switching valve 20 in the direction of flow from the tank 16 to the plunger chamber 26. The check valve 28 also permits further extension of the plunger piston 24 relative to the plunger cylinder housing 22 in the blocking position SS of the switching valve 20, by drawing hydraulic fluid into the plunger chamber 26 via the line arrangement 14 in that the check valve 28 is opened during an extending movement of the plunger piston 24 from the tank 16. Also in this embodiment, the check valve 28 is a spring-loaded check valve 28 and it opens against the spring force in the direction of flow from the tank 16 to the plunger chamber 26.

Secondly, the third embodiment of the hydraulic locking device 10 according to the invention shown in FIG. 4 differs from the embodiment shown in FIG. 3 in that a branch line 30 branches off from the line arrangement 14 between the switching valve 20 and the plunger chamber 26. As shown, the branch line 30 is disposed within the housing 18 such that the branch line 30 opens into the tank. A pressure relief valve 32 is disposed in the branch line, which is a safety valve and protects the high pressure side of the hydraulic locking device 10 against damage. A maximum permissible pressure is set at the pressure relief valve 32. As soon as the pressure in the plunger chamber 26, and thus also at least in portions of the line arrangement 14, exceeds the maximum permissible pressure set at the pressure relief valve 32, the pressure relief valve 32 opens and the plunger chamber 26 is relieved to the tank 16.

Of course, a branch line 30 with a pressure relief valve 32 can also be used in the embodiments of the hydraulic locking device 10 shown in FIGS. 2 and 3. Furthermore, it is also conceivable that the pressure relief valve 32 is provided in the plunger piston 24 as an alternative to or in addition to the check valve 28.

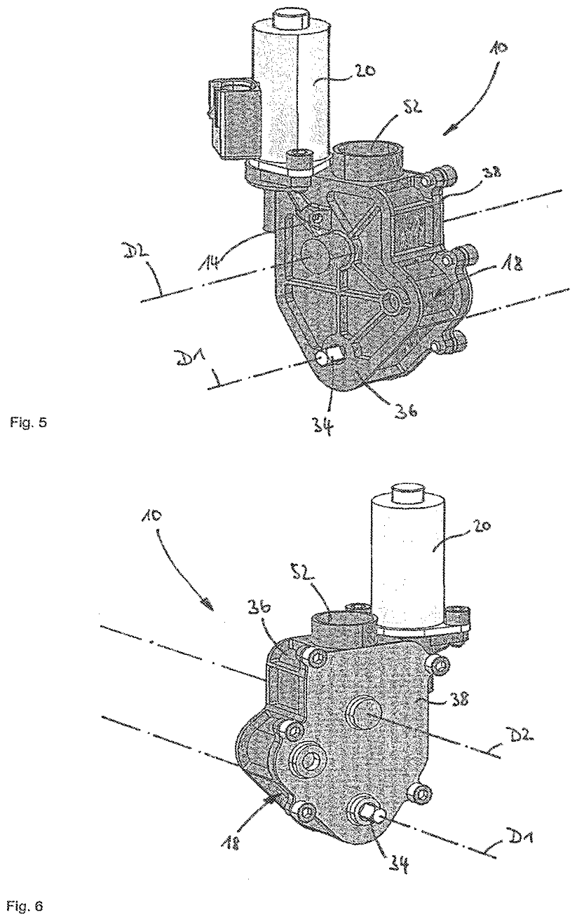

The specific structural design of the hydraulic locking device 10 is explained in more detail below with reference to FIG. 5 to FIG. 9 and FIG. 11. The hydraulic locking device 10 described here corresponds to the embodiment shown in FIG. 3, whereby the following explanations also apply accordingly to the embodiments shown in FIG. 2 and FIG. 4.

The housing 18 of the hydraulic locking device 10 includes a base body 36 and a cover 38. The base body 36 and the cover 38 form an enclosed space within the housing 18, which forms the tank 16. The switching valve 20 is secured to the exterior of the housing, and a portion of the line arrangement 14 also extends from the exterior of the housing 18 into the interior of the housing 18, see FIG. 5. Further, a first receptacle 52 is disposed adjacent the switching valve 20 on the exterior of the housing 18. The first receptacle 52 serves to fix the exoskeleton joint 50 to the exoskeleton, for example by connecting it to a thigh 54 of the exoskeleton, cf. also FIG. 10.

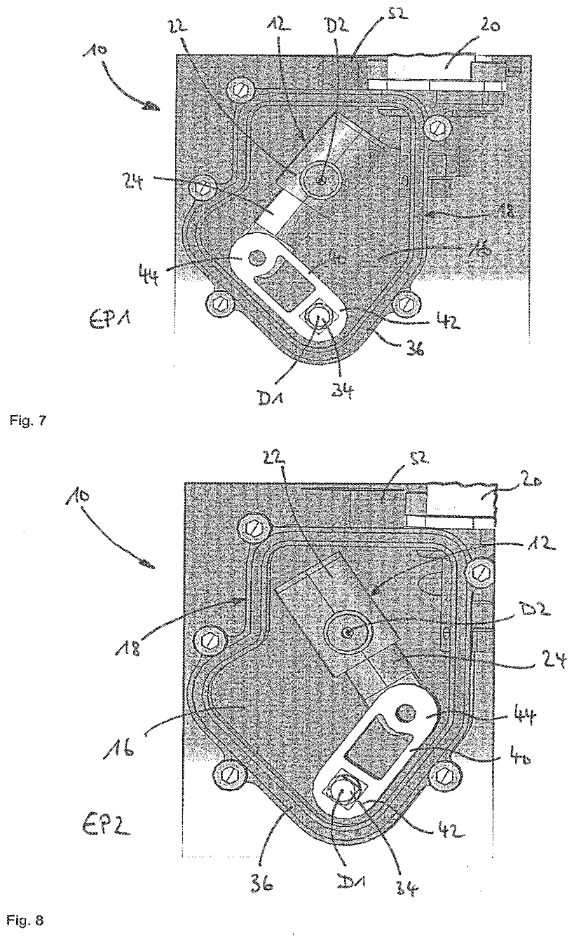

FIGS. 7 to 9 show a side view of the hydraulic locking device 10, with the cover 38 removed. As can be seen, the plunger cylinder 12 is disposed inside the housing 18, namely in the tank 16. Thus, the plunger piston 24 can be sealed with respect to the plunger cylinder housing 22 with a simple seal, for example a gap seal. Any leakage from the plunger chamber 26 exits directly into the tank 16, so that no special means for preventing leakage are necessary.

The hydraulic locking device 10 further includes a rotary shaft 34 extending through the housing 18. Specifically, the rotary shaft 34 extends through the main body 36 and through the cover 38 such that one end of the rotary shaft 34 protrudes from the housing 18 on either side thereof. The rotary shaft 34 is sealed with respect to the housing 18 and the tank 16, respectively, and a relatively simple seal is sufficient since only atmospheric pressure prevails in the tank 16. As shown, a square is provided at each end of the rotary shaft 34 to connect the rotary shaft to further parts in a rotationally fixed manner, cf. FIG. 10. Of course, other possibilities can also be provided here to connect the rotary shaft 34 to the further parts.

The rotary shaft 34 defines a first axis of rotation D1. The plunger cylinder housing 22 is also rotatably disposed within the housing 18. As shown, the plunger cylinder housing 22 is rotatably mounted about a second axis of rotation D2 on the base body 36 and the cover 38. The second axis of rotation D2 is arranged in parallel to the first axis of rotation D1.

A lever 40 is disposed within the housing 18 or the tank 16. The lever 40 has a shaft coupling portion 42 at one end thereof and a piston coupling portion 44 at the other end thereof. The shaft coupling portion 42 is non-rotatably connected to the rotary shaft 34, and the piston coupling portion 44 is rotatably connected to the end of the plunger piston 24 protruding from the plunger cylinder housing 22.

When the rotary shaft 34 rotates about the first axis of rotation D1, the rotary motion is translated into a linear motion of the plunger piston 24 by the rotatable support of the plunger cylinder housing 22 about the second axis of rotation D2 and the lever 40. As shown, the rotary shaft 34 is rotatable between a first end position EP1 (cf. FIG. 7) and a second end position EP2 (cf. FIG. 8). For example, the first end position EP1 may correspond to a fully open position of the exoskeleton joint 50 and the second end position EP2 may correspond to, for example, a fully closed end position EP2 of the exoskeleton joint 50.

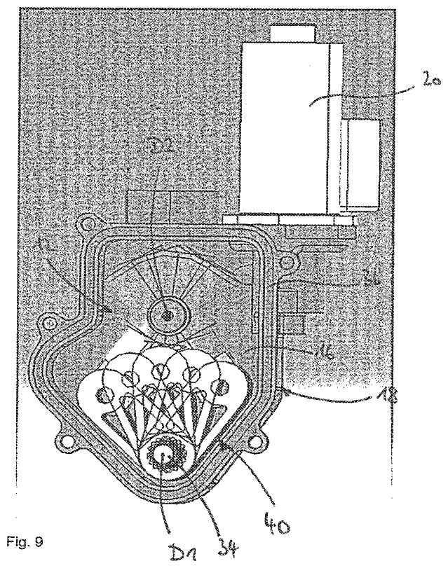

In the first end position EP1 of the rotary shaft 34, the plunger piston 24 is substantially fully extended. Upon rotation of the rotary shaft 34 from the first end position EP1 toward the second end position EP2, the plunger piston 24 retracts and the plunger cylinder housing 22 rotates about the second axis of rotation D2. The plunger piston 24 retracts until a dead point position is reached between the first end position EP1 and the second end position EP2. At this dead point position, the plunger piston 24 is fully retracted although the rotary shaft 34 is not yet in the second end position EP2. Upon further rotation of the rotary shaft 34 about the first axis of rotation D1 from the dead point position toward the second end position EP2, the plunger piston 24 extends again until it is again substantially extended when the rotary shaft 34 is at the second end position EP2. Because of this stroke reversal over the complete movement cycle between the first end position EP1 and the second end position EP2, a particularly compact plunger cylinder 12 can be used. FIG. 9 shows the various positions of the lever 40, the plunger piston 24 and the plunger cylinder housing 22.

As can be seen from the partial sectional view shown in FIG. 11 (the switching valve 20 is not shown in sectional view), the check valve 28 is disposed in the plunger piston 24. For this purpose, the plunger piston 24 has an axial opening 46 for receiving the check valve 28. One end of the axial opening 46 communicates with the plunger chamber 26, and the other end opens into a bore 48 which passes radially through the plunger piston 24 and communicates with the tank 16. When the switching valve 20 is in the blocking position SS, hydraulic fluid can be sucked through the axial opening 46 into the plunger chamber 26 via the bore 48 and the check valve 28, so that extension of the plunger piston 24 is still possible.

FIGS. 10 and 11 show an exoskeleton joint 50 with a hydraulic locking device 10 according to the invention. The exoskeleton joint 50 shown is an exoskeleton knee joint. As shown, a thigh 54 of an exoskeleton is received in the first receptacle 52. Further, the exoskeleton knee joint 50 includes a second receptacle 56 that is movably mounted to the housing 18 via a four-bar link 58. A lower leg 60 of the exoskeleton is fixed to the second receptacle 56 in this embodiment.

The four-bar linkage 58 includes a first leg 62 having one end non-rotatably connected to the rotary shaft 34 and the other end rotatably connected to the second receptacle 56. Further, the four-bar linkage 58 includes a second leg 64 rotatably connected at one end to the housing 18 and rotatably connected at the other end to the second receptacle 56. The four-bar linkage 58 allows the pivot point between the thigh 54 and the lower leg 60 to be offset from the housing 18 and to be optimally selected from an anatomical standpoint.

### LIST OF REFERENCE SIGNS

10 hydraulic locking device
12 plunger cylinder
14 line arrangement
16 tank
18 housing
20 switch valve
22 plunger cylinder housing
23 biasing element
24 plunger piston
26 plunger chamber
27 actuating device
28 check valve
30 branch line
32 pressure relief valve
34 rotary shaft
36 base body
38 cover
40 lever
42 shaft coupling portion
44 piston coupling portion
46 axial opening
48 bore
50 exoskeleton joint/exoskeleton knee joint
52 first receptable
54 thigh
56 second receptable
58 four-bar linkage
60 lower leg
62 first leg
64 second leg
100 hydraulic locking device
102 hydraulic cylinder/differential cylinder
104 line arrangement
106 accumulator/tank
108 cylinder housing
110 piston
112 piston chamber
114 rod chamber
116 first branch
118 second branch
120 switching valve
122 short-circuit line
124 check valve
D1 first axis of rotation
D2 second axis of rotation
EP1 first end position
EP2 second end position
FS release position
SS blocking position

1. A hydraulic locking device for an exoskeleton joint, comprising:
a housing;
an extendable and retractable hydraulic cylinder;
a tank; and
a switching valve,
wherein the hydraulic cylinder is connected to the tank via a line arrangement and the switching valve is disposed in the line arrangement,
wherein the switching valve is configured to be switched between a release position and a blocking position,
wherein the hydraulic cylinder is freely movable in the release position of the switching valve and the switching valve prevents retraction of the hydraulic cylinder in the blocking position,
wherein the hydraulic cylinder is a plunger cylinder with a plunger cylinder housing and a plunger piston movably disposed in the plunger cylinder housing,
wherein the plunger cylinder is disposed within the housing.

2. The hydraulic locking device according to claim 1, wherein the hydraulic locking device has a rotary shaft and a lever,
wherein the rotary shaft is rotatably mounted about a first axis of rotation on the housing and the plunger cylinder housing is rotatably mounted about a second axis of rotation on the housing, and
the lever comprises a shaft coupling portion and a piston coupling portion, the shaft coupling portion being non-rotatably connected to said rotary shaft, and
wherein the piston coupling portion is rotatably connected to the plunger.

3. The hydraulic locking device according to claim 2, wherein the rotary shaft is rotatable between a first end position and a second end position,
wherein a dead point position is located between the first end position and the second end position,
wherein the plunger is extended in the first end position and enters the dead point position upon rotation of the rotary shaft and extends upon rotation of the rotary shaft from the dead point position to the second end position.

4. The hydraulic locking device according to claim 1, wherein the switching valve has a check valve which is active in the blocking position, the check valve opening in the direction of flow from the tank to the plunger cylinder.

5. The hydraulic locking device according to claim 1, wherein the plunger cylinder has a check valve, the check valve opening in the direction of flow from the tank to the plunger cylinder.

6. The hydraulic locking device according to claim 5, the plunger comprises the check valve.

7. The hydraulic locking device according to claim 1, the plunger piston and the plunger cylinder housing define a plunger chamber, the plunger chamber being connected to the tank via a pressure relief valve.

8. The hydraulic locking device according to claim 1, wherein the housing forms said tank, wherein the plunger cylinder s disposed within the tank.

9. An exoskeleton joint comprising a hydraulic locking device according to claim 1.

10. The exoskeleton joint according to claim 9, wherein the exoskeleton joint has a first receptacle disposed on the housing and a second receptacle movably mounted on the housing via a four-bar linkage.
